(19) United States
(12) Patent Application Publication
(10) Pub. No.: US 2024/0041747 A1
(43) Pub. Date: Feb. 8, 2024

(54) AQUEOUS COSMETIC

(71) Applicant: L V M H RECHERCHE, Saint-Jean-De-Braye (FR)

(72) Inventors: Koichi KISHIDA, Chiyoda-ku, Tokyo (JP); Mai OZAWA, Chiyoda-ku, Tokyo (JP)

(73) Assignee: L V M H RECHERCHE, Saint-Jean-De-Braye (FR)

(21) Appl. No.: 18/268,690

(22) PCT Filed: Dec. 22, 2020
(86) PCT No.: PCT/IB2020/001136

§ 371 Date: Jun. 21, 2023

(51) Int. Cl. A61K 8/73; A61Q 19/00

(52) U.S. Cl. CPC A61K 8/73; A61Q 19/00; A61K 2800/10; A61K 2800/80

(57) ABSTRACT

An aqueous cosmetic comprising pullulan, a polysaccharide including a uronic acid and a cyclic aldohexose compound as constituent monosaccharides, an amphiphilic substance, and an acrylates/C₁₀₋₃₀ alkyl acrylate crosspolymer.

### TECHNICAL FIELD

The present invention relates to an aqueous cosmetic.

### BACKGROUND ART

A large variety of cosmetics that enhance skin texture during use and after application have been proposed in the past.

Japanese Patent Publication No. 2018-165258, for example, discloses a moisturizing cosmetic with smooth texture and non-stickiness comprising pullulan, a polyhydric alcohol such as 1,3-propanediol, and water, wherein the mass ratio of the polyhydric alcohol with respect to the pullulan is 50 to 250 and the mass ratio of 1,3-propanediol with respect to the polyhydric alcohol is of 0.2 to 1. Japanese Patent Publication No. 2018-39790 discloses a composition comprising polyglyceryl-10 eicosanedioate/tetradecanedioate, tripropylene glycol and an oil agent having a specific gravity of 0.80 to 0.95.

### Technical Problem

Aqueous cosmetics typically have watery textures and are used for a variety of purposes including lotions and essences. However, adding a resilient and supple sensation to the watery texture of an aqueous cosmetic, for example by adding pullulan or polyols in high content, has tended to produce a sticky feel.

So there is still a need to provide an aqueous cosmetic having an excellent resilient and supple sensation and watery texture during application, while also inhibiting stickiness after application (also named ‘non-stickiness’) and the present invention specifically answers these expectations by combining a pullulan, a polysaccharide including a uronic acid and a cyclic aldohexose compound as constituent monosaccharides, an amphiphilic substance, and an acrylates/C₁₀₋₃₀ alkyl acrylate crosspolymer.

### SUMMARY OF THE INVENTION

One aspect of the invention relates to an aqueous cosmetic comprising pullulan, a polysaccharide including a uronic acid and a cyclic aldohexose compound as constituent monosaccharides, an amphiphilic substance, and an acrylates/C₁₀₋₃₀ alkyl acrylate crosspolymer.

In a particular embodiment, the polysaccharide is a Tremella fuciformis polysaccharide. In a particular embodiment, the amphiphilic substance includes one or more compounds selected from the group consisting of polyglycerol fatty acid esters and polyoxyalkylene glyceryl ethers, and in particular includes polyglyceryl-10 eicosanedioate/tetradecanedioate.

The aqueous cosmetic may further comprise a polyol. The polyol may also include one or more selected from the group consisting of glycerin, butylene glycol, propanediol, pentylene glycol and polyoxyalkylenemethyl glucosides.

The present invention also relates to a cosmetic process for caring keratinic materials, comprising the application onto keratinic materials, in particular onto skin, of the cosmetic composition of the invention. The cosmetics of the present invention include skin-care cosmetics, in particular anti-aging cosmetics but may also include moisturizing cosmetics, cleansing cosmetics, or whitening cosmetics.

### DETAILED DESCRIPTION OF THE INVENTION

Embodiments of the invention will now be described. However, the present invention is not limited to the embodiments described below.

According to one embodiment, the aqueous cosmetic comprises (a) pullulan (hereunder also referred to as “component (a)”), (b) a polysaccharide including a uronic acid and a cyclic aldohexose compound as constituent monosaccharides (hereunder also referred to as “component (b)”), (c) an amphiphilic substance (hereunder also referred to as “component (c)” and (d) an acrylates/C₁₀₋₃₀ alkyl acrylate crosspolymer (hereunder also referred to as “component (d)”). Throughout the present specification, “aqueous cosmetic” refers to a cosmetic that includes water and is in a non-emulsified state (not an emulsion).

### Pullulan

The pullulan (component (a)) is a straight-chain glucan in which a single unit is maltotriose having three α-1,4-bonded glucose molecules, with each being bonded to the next maltotriose by repeated α-1,6 bonding. The pullulan gives resilient and supple sensation to provide anti-wrinkle effect.

In a particular embodiment, the content of component (a) is 0.1 mass % or greater, 0.2 mass % or greater, 0.3 mass % or greater, greater than 0.3 mass % or 0.4 mass % or greater, and 5.0 mass % or lower, 4.0 mass % or lower, 3.0 mass % or lower, 2.0 mass % or lower, 1.5 mass % or lower, 1.0 mass % or lower, 0.8 mass % or lower or 0.6 mass % or lower, based on the total mass of the aqueous cosmetic. In a particular embodiment, the content of component (a) ranges from 0.1 mass % to 5.0 mass %, in particular from 0.2 mass % to 3.0 mass % or 0.3 mass % to 1.0 mass %, based on the total mass of the aqueous cosmetic.

Examples for component (a) may be any commercially available pullulan compounds, used directly, in particular the “Cosmetic Pullulan”, which is supplied by Hayashibara Co., Ltd.

### Polysaccharides

Component (b) is a polysaccharide including a uronic acid and a cyclic aldohexose compound as constituent monosaccharides. Component (b) also functions as a thickener. The polysaccharide may also be biologically derived. Biologically derived polysaccharides according to the invention are polysaccharides extracted from plants (plant-derived) or fungi (fungi-derived, such as from Basidiomycota or Ascomycota), by extraction processes. The term “biologically derived” also means a compound that has been purified and processed from a substance (such as a polymer substance) produced by a microorganism. The constituent monosaccharides may be in the L-form or D-form. Component (b) may be a single polysaccharide used alone, or a combination of two or more polysaccharides may be used.

Uronic acids are sugar oxidation products, being saccharide derivatives with carboxy groups. Examples of uronic acids include glucuronic acid, galacturonic acid, mannuronic acid, arabinonic acid, fructuronic acid, tagaturonic acid, iduronic acid and guluronic acid. The polysaccharide may contain a single type of uronic acid, or it may include a combination of two or more different types of uronic acids. In a particular embodiment, the uronic acid is preferably glucuronic acid or galacturonic acid, or mixture thereof.

The cyclic aldohexose compound may be pyranose having a six-membered ring composed of 5 carbon atoms and one oxygen atom. The cyclic aldohexose compound may include one or more selected from the group consisting of allose, altrose, glucose, mannose, gulose, idose, galactose and talose. In a particular embodiment, the cyclic aldohexose compound includes glucose or mannose, or mixture thereof.

In a particular embodiment, component (b) may also include one of the aforementioned cyclic aldohexose compounds and another constituent monosaccharide other than a uronic acid. Other constituent monosaccharides may include cyclic deoxyaldohexose compounds and/or cyclic aldopentose compounds.

A cyclic deoxyaldohexose compound is preferably pyranose having a cyclic structure derived from deoxyaldohexose, i.e. a six-membered ring composed of 5 carbon atoms and one oxygen atom. In a particular embodiment, cyclic deoxyaldohexose compounds include fucose, fuculose and rhamnose, and mixtures thereof. The cyclic deoxyaldohexose compound may include a single type alone, or it may include a combination of two or more types. In a particular embodiment, the cyclic deoxyaldohexose compound is fucose (such as L-fucose), or a mixture of fucose and rhamnose.

A cyclic aldopentose compound is a cyclic pentose having a six-membered ring composed of 5 carbon atoms and one oxygen atom or a five-membered ring composed of 4 carbon atoms and one oxygen atom, derived from aldopentose. In a particular embodiment, cyclic aldopentose compounds include ribose, arabinose, xylose and lyxose, and mixtures thereof. The cyclic aldopentose compound may include a single type alone, or it may include a combination of two or more types. The cyclic aldopentose compound is preferably xylose. So, in a particular embodiment, other constituent monosaccharides include a cyclic aldopentose compound (preferably a xylose) or a cyclic deoxyaldohexose compound (preferably a fucose).

In a particular embodiment, component (b) includes (i) a uronic acid, (ii) a cyclic aldohexose compound and (iii) a cyclic deoxyaldohexose compound or cyclic aldopentose compound, as constituent monosaccharides. In a particular embodiment, component (b) includes (i) to (iii) as major constituent monosaccharides, and it may also include other constituent monosaccharides in addition to (i) and (iii). The total proportion of (i), (ii) and (iii) in component (b) may be 50 mol % or greater, for example, with respect to the total moles of constituent monosaccharides.

In a particular and preferred embodiment, component (b) includes galacturonic acid, galactose and fucose (such as L-fucose), or it includes glucuronic acid, mannose and xylose, as constituent monosaccharides.

In a particular and preferred embodiment, component (b) includes one or more selected from the group consisting of Tremella fuciformis polysaccharides and biosaccharide gum-1.

Tremella fuciformis polysaccharides are polysaccharides extracted from Tremella fuciformis. Tremella fuciformis polysaccharides are fungi-derived polysaccharides with glucuronic acid, mannose and xylose as constituent monosaccharides. Tremella fuciformis polysaccharides have a main chain with mannose as the constituent monosaccharide and side chains with xylose and glucuronic acid as structural units. The molar percentage of glucuronic acid with respect to the number of moles of constituent monosaccharides is about mol %. Examples of Tremella fuciformis polysaccharides include the trade name Tremoist® TP (INCI name: Tremella fuciformis polysaccharide) (product of Nippon Seika Co., Ltd.). The average molecular weight of Tremoist® TP is 1,000,000 or greater.

Biosaccharide gum-1 is a sorbitol fermented polysaccharide, obtained from sorbitol. Biosaccharide gum-1 is a polysaccharide having galacturonic acid, galactose and L-fucose as constituent monosaccharides. An example of a biosaccharide gum-1 is the commercial material FUCOGEL® (product of Nikko Chemicals Co., Ltd.).

In a particular embodiment, the total content of component(s) (b) is 0.001 mass % or greater, 0.005 mass % or greater, 0.010 mass % or greater, greater than 0.015 mass %, and 1.0 mass % or lower, 0.5 mass % or lower, 0.1 mass % or lower, 0.08 mass % or lower, 0.06 mass % or lower, 0.04 mass % or lower or 0.03 mass % or lower, based on the total mass of the aqueous cosmetic. In a particular embodiment, the total content of component(s) (b) ranges from 0.001 mass % to 1.0 mass %, in particular from 0,005 mass % to 0.10 mass % or 0.010 mass % to 0.05 mass %, based on the total mass of the aqueous cosmetic.

### Amphiphilic Substance

The amphiphilic substance (component (c)) is a compound having a hydrophilic group and a hydrophobic group in the same molecule. Examples of hydrophilic groups include hydroxy groups and polyoxyalkylene groups such as polyoxyethylene. Examples of hydrophobic groups include hydrocarbon groups such as long-chain alkyl groups.

In a composition comprising pullulan, stickiness during application will tend to increase due to the hydrophilic groups in the pullulan. In the present invention, the stickiness is more likely to be inhibited through the use of an amphiphilic substance.

In a particular embodiment, component (c) is a polyglycerol fatty acid ester or polyoxyalkylene glyceryl ether, for example. An amphiphilic substance may be used alone as a single type, or two or more types may be used in combination.

Examples of polyglycerol fatty acid esters include polyglyceryl-10 eicosanedioate/tetradecanedioate (such as Neosolue™-AquaS (product of Nippon Seika Co., Ltd.) and polyglyceryl-10 laurate (such as S-FACE 10G-L, product of Sakamoto Yakuhin Kogyo Co., Ltd.).

In a particular embodiment, polyoxyalkylene glyceryl ethers include polyethylene glycol (PEG)/polypropylene glycol (PPG)/polybutylene glycol-8/5/3 glycerin (for example, WILBRIDE^{R} S-753D (by NOF Corp.), or PPG-24 GLYCERES-24 (such as Nikkol SG-G2424 by Nikko Chemicals Co., Ltd.).

From the viewpoint of obtaining an even more excellent effect of the invention, component (c) may include one or more selected from the group consisting of polyglycerol fatty acid esters and polyoxyalkylene glyceryl ethers, while from the viewpoint of further inhibiting stickiness after application, a polyglycerol fatty acid ester is even more preferred, in particular polyglyceryl-10 eicosanedioate/tetradecanedioate.

In a particular embodiment, the total content of component(s) (c) is 0.5 mass % or greater, 1.0 mass % or greater, 1.5 mass % or greater, greater than 2.0 mass % or 2.5 mass % or greater, and 10.0 mass % or lower, 8.0 mass % or lower, 6.0 mass % or lower or 4.0 mass % or lower, based on the total mass of the aqueous cosmetic. In a particular embodiment, the total content of component(s) (c) ranges from 0.5 mass % to 10.0 mass %, in particular from 1.0 mass % to 5.0 mass %, based on the total mass of the aqueous cosmetic.

### Acrylates/C₁₀₋₃₀ Alkyl Acrylate Crosspolymer

In a particular embodiment, the acrylates/C₁₀₋₃₀ alkyl acrylate crosspolymer (component (d)) is a copolymer of one or more monomers selected from the group consisting of acrylic acid, methacrylic acid and their esters, and an alkyl acrylate having an alkyl group of 10 to 30 carbon atoms, wherein the sucrose allyl ether or the pentaerythritol allyl ether is crosslinked.

Examples of commercial products for component (d) include PEMULEN™ TR-1 (by Lubrizol), CARBOPOL® ULTREZ 20 (by Lubrizol) and PEMULEN™ EZ-4U (by Lubrizol).

In a particular embodiment, the total content of component(s) (d) is 0.01 mass % or greater, 0.05 mass % or greater, 0.10 mass % or greater, or 0.15 mass % or greater, and 5.0 mass % or lower, 3.0 mass % or lower, 1.0 mass % or lower, 0.5 mass % or lower or 0.3 mass % or lower, based on the total mass of the aqueous cosmetic. In a particular embodiment, the total content of component(s) (d) ranges from 0.01 mass % to 5.0 mass %, in particular from 0.05 mass % to 1.0 mass % or mass % to 0.5 mass %, based on the total mass of the aqueous cosmetic.

### Polyols

In a particular and preferred embodiment, the aqueous cosmetic comprises at least one or more polyol(s). A polyol is a compound having two or more hydroxy groups. The number of hydroxy groups of the polyol may be 2 to 10 or 2 to 8, for example. The polyol may be of a single type alone, or it may include a combination of two or more types. The polyol may also include two or more polyols with different numbers of hydroxy groups. The polyol may include one or more selected from the group consisting of diols which have two hydroxy groups (such as butylene glycol, propanediols pentylene glycol, 1,2-hexanediol and caprylyl glycol), triols which have three hydroxy groups (such as glycerin) and tetraols which have four hydroxy groups, or it may include a diol, triol and tetraol.

Examples of polyols include glycerin, butylene glycol, propanediols (such as 1,2-propanediol and 1,3-propanediol), pentylene glycol (1,2-pentanediol), polyoxyalkylenemethyl glucosides, 1,2-hexanediol and caprylyl glycol. Examples of polyoxyalkylenemethyl glucosides include methylgluces-20 and methylgluces-10. In a particular embodiment, the composition comprises at least a triol and one or more diols, in particular comprises at least glycerin as triol and one or more diols selected in the group consisting of butylene glycol, propanediols (such as 1,2-propanediol and 1,3-propanediol), pentylene glycol (1,2-pentanediol), and preferably mixtures thereof.

In a particular embodiment, the total content of polyol(s) is 5 mass % or greater, 10 mass % or greater, 15 mass % or greater or 20 mass % or greater, and 40 mass % or lower, 35 mass % or lower, 30 mass % or lower or 25 mass % or lower, based on the total mass of the aqueous cosmetic. In a particular embodiment, the total content of polyol(s) ranges from 5 mass % to 40 mass %, in particular 10 mass % to or 15 mass % to 30 mass %, or 20 mass % to 25 mass % based on the total mass of the aqueous cosmetic.

The triol content may be 5 mass % or greater or 8 mass % or greater, based on the total mass of the aqueous cosmetic, and from the viewpoint of more easily obtaining a supple sensation during application of the composition, it may be greater than 10 mass % or 12 mass % or greater. The triol content may be 25 mass % or lower, 20 mass % or lower or 15 mass % or lower, based on the total mass of the aqueous cosmetic. The triol may be of a single type alone, or it may include a combination of two or more types. In a particular embodiment, the total content of triol(s) ranges from 5 mass % to 25 mass %, in particular from 10 mass % to 20 mass % or 12 mass % to 15 mass %, based on the total mass of the aqueous cosmetic. In a particular and preferred embodiment, the content of glycerin ranges from 10 mass % to 20 mass %, preferably from 12 mass % to 15 mass %, based on the total mass of the aqueous cosmetic.

The diol content may be 5 mass % or greater or 8 mass % or greater, based on the total mass of the aqueous cosmetic. The diol content may be 20 mass % or lower or 15 mass % or lower, based on the total mass of the aqueous cosmetic, and from the viewpoint of more easily obtaining a supple sensation during application of the composition, it may be 13 mass % or lower or 10 mass % or lower. The diol may be of a single type alone, or it may include a combination of two or more types. In a particular embodiment, the total content of diol(s) ranges from 5 mass % to 20 mass %, in particular from 8 mass % to 13 mass %, based on the total mass of the aqueous cosmetic.

The tetraol content may be 0.1 mass % or greater or 0.3 mass % or greater, based on the total mass of the aqueous cosmetic. The tetraol content may be 5 mass % or lower, 3 mass % or lower or 1 mass % or lower, based on the total mass of the aqueous cosmetic. The tetraol may be of a single type alone, or it may include a combination of two or more types. In a particular embodiment, the total content of tetraol(s) ranges from 0.1 mass % to 5 mass %, in particular from 0.3 mass % to 3 mass %, based on the total mass of the aqueous cosmetic.

The aqueous cosmetic will generally include water at 50 mass % or greater based on the total mass of the aqueous cosmetic. The total water content of the aqueous cosmetic may be 55 mass % or greater, 60 mass % or greater, 65 mass % or greater or 70 mass % or greater, and 95 mass % or lower, 90 mass % or lower, 85 mass % or lower or 80 mass % or lower, based on the total mass of the aqueous cosmetic. In a particular embodiment, the total water content ranges from 50 mass % to 95 mass %, in particular from 55 mass % to 80 mass % or from 60 mass % to 75 mass %, based on the total mass of the aqueous cosmetic.

The aqueous cosmetic may further comprise other components in addition to those mentioned above. Such other components include microbicides (such as chlorphenesin), monools, oil agents, perfumes and pH adjustors, and mixtures thereof. An example of a monool is PPG-26-Buteth-26. An example of an oil agent is PEG-40 hydrogenated castor oil. The content of other components may be 0.1 mass % or greater, 0.5 mass % or greater, 1.0 mass % or greater or 1.5 mass % or greater, and mass % or lower, 3.0 mass % or lower or 2.0 mass % or lower, based on the total mass of the aqueous cosmetic. In a particular embodiment, the total content of other components ranges from 0.1 mass % to 10 mass %, based on the total mass of the aqueous cosmetic.

The aqueous cosmetic of the embodiment has an excellent resilient and supple sensation and watery texture during application, without stickiness after application, and it can be also transparent. In a particular embodiment, the aqueous cosmetic of the invention is used by being filled into an ampule container.

The aqueous cosmetic can be produced by a method that includes mixing each of the components mentioned above, for example. The components may be mixed either simultaneously or separately. Mixture of the components mentioned above may be in any desired order. They may also be optionally mixed while heating.

The aqueous cosmetics of each of the embodiments described above are particularly suitable for use in lotions (cosmetic water), essences and masks (for example facial masks). In particular, the aqueous cosmetic of the present invention include skin care cosmetics such as anti-aging cosmetics (such as anti-wrinkle cosmetics), cleansing cosmetics, moisturizing cosmetics, or whitening cosmetics.

### Cosmetic Process

The invention also relates to a cosmetic process for caring keratinic materials, comprising the application onto keratinic materials, in particular onto skin, of the cosmetic composition of the invention. In particular, the composition is applied on the face and/or hands.

### Examples

The invention will now be illustrated by the following examples, with the understanding that the invention is not meant to be limited to these examples.

<Preparation of Aqueous Cosmetic>

Chlorphenesin was dissolved in water at 80° C. to prepare component (A1) comprising chlorphenesin and water. The components listed in Table 1 were dispersed with a spatula to prepare component (B) and component (E).

Component (A1) was added to a mixing tank. Component (A2) of each of the compositions listed in Table 1 was gradually added to the mixing tank and stirred at room temperature for 15 minutes. Component (A3) of the compositions listed in Table 1 was then added to the mixing tank and the temperature was raised to 80° C. while stirring. The mixture of component (A1), component (A2) and component (A3) was stirred for 30 minutes. The liquid mixture was then air-cooled to 35° C. Next, component (B) was added to the mixing tank under 70° C. conditions. Component (C) of the compositions listed in Table 1 was added to the mixing tank under 50° C. conditions. All of the remaining components listed in Table 1 were then added to and stirred in the mixing tank under 40° C. conditions. After stirring, the mixture was cooled to 35° C. to obtain the final aqueous cosmetic.

Comparatives examples are as followed:
Comparative 1: without component (d) of the invention
Comparative 2: without component (d) of the invention
Comparative 3: without component (a) of the invention
Comparative 4: without component (b) of the invention
Comparative 5: without component (b) of the invention
Comparative 6: without component (b) of the invention

<Organoleptic Evaluation>

Each of the aqueous cosmetics of the Examples and Comparative Examples were organoleptically evaluated for resilient and supple sensation, non-stickiness after application and watery texture during application. For the organoleptic evaluation, each aqueous cosmetic was coated onto skin by a cosmetic expert evaluation panel, with evaluation being conducted based on the following scale.

An evaluation of “A” or “B” for all of the evaluations for resilient and supple sensation, non-stickiness after application and watery texture during application was judged to be an excellent evaluation.

(1) Resilient and Supple Sensation
A: Strong
B: Moderate
C: Almost none
D: None

(2) Non-Stickiness after Application
A: Absolutely no stickiness
B: Almost no stickiness
C: Some stickiness
D: Stickiness

(3) Watery Texture During Application
A: Strong
B: Moderate
C: Almost none
D: None

TABLE 1

| | | | Example | | | | | Comparative Example | | | | | |
|---|---|---|---|---|---|---|---|---|---|---|---|---|---|
| Phase | | | 1 | 2 | 3 | 4 | 5 | 1 | 2 | 3 | 4 | 5 | 6 |
| A1 | Aqua | | 70.09 | 68.09 | 70.59 | 70.09 | 70.29 | 70.09 | 69.79 | 70.59 | 70.09 | 70.11 | 71.11 |
| | Chlorphenesin | | 0.27 | 0.27 | 0.27 | 0.27 | 0.27 | 0.27 | 0.27 | 0.27 | 0.27 | 0.27 | 0.27 |
| A2 | Pullulan | | 0.5 | 0.5 | 0.5 | 0.5 | 0.3 | 0.5 | 0.5 | — | 0.5 | 0.5 | 0.5 |
| | (cosmetic grade) | | | | | | | | | | | | |
| | component (a) | | | | | | | | | | | | |
| A3 | Polymer | Acrylates/ | 0.2 | 0.2 | 0.2 | 0.2 | 0.2 | — | — | 0.2 | 0.2 | 0.2 | 0.2 |
| | Component | C10-30 | | | | | | | | | | | |
| | (d) | alkyl | | | | | | | | | | | |
| | | acrylate | | | | | | | | | | | |
| | | crosspolymer | | | | | | | | | | | |
| | | (CARBOPOL ® | | | | | | | | | | | |
| | | ULTREZ 20) | | | | | | | | | | | |
| | | invention | | | | | | | | | | | |
| | | Carbomer | — | — | — | — | — | 0.2 | — | — | — | — | — |
| | | (CARBOPOL ® | | | | | | | | | | | |
| | | ULTREZ 30 | | | | | | | | | | | |
| | | POLYMER | | | | | | | | | | | |
| | | (EX-1142)) | | | | | | | | | | | |
| | | comparative | | | | | | | | | | | |
| | | Polyacrylate | — | — | — | — | — | — | 0.5 | — | — | — | — |
| | | crosspolymer-6 | | | | | | | | | | | |
| | | (SEPIMAX | | | | | | | | | | | |
| | | ZEN ™) | | | | | | | | | | | |
| | | comparative | | | | | | | | | | | |
| B | Poly- | Tremella | 0.02 | 0.02 | 0.02 | 0.02 | 0.02 | 0.02 | 0.02 | 0.02 | — | — | — |
| | saccharides | fuciformis | | | | | | | | | | | |
| | Component | polysaccharide | | | | | | | | | | | |
| | (b) | (TREMOIST TP) | | | | | | | | | | | |
| | Polyols | invention | | | | | | | | | | | |
| | | Sodium | — | — | — | — | — | — | — | — | 0.02 | — | 1 |
| | | acetylated | | | | | | | | | | | |
| | | hyaluronate | | | | | | | | | | | |
| | | comparative | | | | | | | | | | | |
| | | Glycerin | 14 | 14 | 14 | 10 | 14 | 14 | 14 | 14 | 14 | 14 | 14 |
| | | Butylene glycol | 5 | 5 | 5 | 5 | 5 | 5 | 5 | 5 | 5 | 5 | 5 |
| | | Propanediol | 3 | 3 | 3 | 7.5 | 3 | 3 | 3 | 3 | 3 | 3 | 3 |
| | | Pentylene glycol | 1 | 1 | 1 | 1 | 1 | 1 | 1 | 1 | 1 | 1 | 1 |
| | | Methyl | 0.5 | 0.5 | 0.5 | 0.5 | 0.5 | 0.5 | 0.5 | 0.5 | 0.5 | 0.5 | 0.5 |
| | | gluceth-20 | | | | | | | | | | | |
| | Amphiphilic | Polyglyceryl-10 | 3 | — | 2.5 | 2.5 | 3 | 3 | 3 | 3 | 3 | 3 | 2 |
| | substances | eicosanedioate/ | | | | | | | | | | | |
| | Component | tetradecanedioate, | | | | | | | | | | | |
| | (c) | glycerin | | | | | | | | | | | |
| | | (NEOSOLUE- | | | | | | | | | | | |
| | | AQUA S) | | | | | | | | | | | |
| | | PEG/PPG/ | 1 | 5 | — | — | — | — | — | — | — | — | — |
| | | Poly- | | | | | | | | | | | |
| | | butyleneglycol- | | | | | | | | | | | |
| | | 8/5/3 Glycerin | | | | | | | | | | | |
| | | (WILBRIDE | | | | | | | | | | | |
| | | S-753D) | | | | | | | | | | | |
| C | Aqua | | 0.5 | 0.5 | 0.5 | 0.5 | 0.5 | 0.5 | 0.5 | 0.5 | 0.5 | 0.5 | 0.5 |
| | Sodium hydroxide | | 0.04 | 0.04 | 0.04 | 0.04 | 0.04 | 0.04 | 0.04 | 0.04 | 0.04 | 0.04 | 0.04 |
| D | Colorant | | 0.33 | 0.33 | 0.33 | 0.33 | 0.33 | 0.33 | 0.33 | 0.33 | 0.33 | 0.33 | 0.33 |
| E | PPG-26-Buteth-26, PEG-40 | | 1.2 | 1.2 | 1.2 | 1.2 | 1.2 | 1.2 | 1.2 | 1.2 | 1.2 | 1.2 | 1.2 |
| | Hydrogenated castor oil, water | | | | | | | | | | | | |
| | Fragrance | | 0.35 | 0.35 | 0.35 | 0.35 | 0.35 | 0.35 | 0.35 | 0.35 | 0.35 | 0.35 | 0.35 |
| Total | | | 100 | 100 | 100 | 100 | 100 | 100 | 100 | 100 | 100 | 100 | 100 |
| Evaluation | | Resilient | A | A | A | A | B | A | A | D | A | A | A |
| | | and | | | | | | | | | | | |
| | | supple | | | | | | | | | | | |
| | | sensation | | | | | | | | | | | |
| | | Non- | A | B | B | A | A | C | C | A | C | C | D |
| | | stickiness | | | | | | | | | | | |
| | | Watery texture | A | A | A | B | A | B | C | A | C | C | C |

All of Examples 1 to 5 exhibited excellent results for resilient and supple sensation, non-stickiness after application and watery texture during application, in comparison to the Comparative examples. These results demonstrate that the combination of b) a polysaccharide including a uronic acid and a cyclic aldohexose compound as constituent monosaccharides, c) an amphiphilic substance, and d) an acrylates/C₁₀₋₃₀ alkyl acrylate crosspolymer, in an aqueous composition comprising a) pullulan permits achieve excellent results for resilient and supple sensation, non-stickiness after application and watery texture during application.

1. An aqueous cosmetic comprising:
pullulan
a polysaccharide including a uronic acid and a cyclic aldohexose compound as constituent monosaccharides,
an amphiphilic substance, and
an acrylates/C₁₀₋₃₀ alkyl acrylate crosspolymer.

2. The aqueous cosmetic according to claim 1, wherein the polysaccharide is a Tremella fuciformis polysaccharide.

3. The aqueous cosmetic according to claim 1, wherein the amphiphilic substance comprises at least one selected from the group consisting of polyglycerol fatty acid esters and polyoxyalkylene glyceryl ethers.

4. The aqueous cosmetic according to claim 1, wherein the amphiphilic substance is polyglyceryl-10 eicosanedioate/tetradecanedioate.

5. The aqueous cosmetic according to claim 1, which further comprises a polyol.

6. The aqueous cosmetic according to claim 5, wherein the polyol is at least one selected from the group consisting of glycerin, butylene glycol, propanediol, pentylene glycol and polyoxyalkylenemethyl glucosides.

7. The aqueous cosmetic according to claim 5, wherein the total content of polyol(s) ranges from 10 mass % to 35 mass % based on the total mass of the aqueous cosmetic.

8. The aqueous cosmetic composition according to claim 1, wherein the content of water ranges from 50 mass % to mass %, based on the total mass of the aqueous cosmetic.

9. The aqueous cosmetic composition according to claim 1, wherein it is in a form of lotion, essence, or mask.

10. A cosmetic process for caring keratinic materials, comprising the application onto keratinic materials, of the cosmetic composition of claim 1.

11. The cosmetic process according to claim 10, wherein the cosmetic composition is applied onto skin.

12. The aqueous cosmetic according to claim 5, wherein the total content of polyol(s) ranges from 15 mass % to 30 mass % based on the total mass of the aqueous cosmetic.

13. The aqueous cosmetic according to claim 5, wherein the total content of polyol(s) ranges from 20 mass % to 25 mass % based on the total mass of the aqueous cosmetic.
